(19) United States
(12) Patent Application Publication
(10) Pub. No.: US 2014/0343590 A1
(43) Pub. Date: Nov. 20, 2014

(54) Device, And A Method For Treatment Of Increased Blood Pressure

(71) Applicant: Syntach AG, Schaffhause (CH)

(72) Inventors: Jan Otto Solem, Bjarred (SE); Ib Erling Joergensen, Haigerloch (DE)

(21) Appl. No.: 14/344,584

(22) PCT Filed: Sep. 17, 2012
(86) PCT No.: PCT/EP2012/068266

§ 371 Date: Mar. 12, 2014

Related U.S. Application Data
(60) Provisional application No. 61/535,737, filed on Sep. 16, 2011.
(60) Provisional application No. 61/682,824, filed on Aug. 14, 2012.

(51) Int. Cl. A61B 17/34

(52) U.S. Cl. CPC A61B 17/3417; A61B 2017/3454; A61B 2017/00778
USPC 606/185

(57) ABSTRACT

Methods and devices are presented which act by cutting the renal nerves slowly through. The minimally invasive implantable devices can be delivered through state of the art delivery catheters into the renal arteries as known from stenting, and do not require external generators, thereby being easily to introduce in normal equipped radiology catheter labs.

### FIELD OF THE INVENTION

The present invention relates to treatment of increased blood pressure, specifically, to a tissue cutting device and a method for treating such disorders.

### BACKGROUND OF INVENTION

It is well-known that the sympathetic nerves running along (primarily inside) the adventitia of the renal arteries are very influential on systemic hypertension. Several methods treating hypertension by targeting those sympathetic at the renal arteries are known. Neuromodulating agents given as medicine orally (aldosterone receptor blocker among others) are commonly used. Minimally invasive procedures are also known: US2011/0104061 (K. P. Seward), US2011/0184337 (M. A: Evans et al) and US 2008/0213331 A1 (M. Gelfand) describe methods where the nerve activity is reduced or blocked by delivering neurotoxic or nerve-blocking agents locally into the renal artery adventitia. Furthermore methods using vascular ablation catheters by applying temperature, radiofrequency or cryogenic treatment are known.

The neuromodulating agents are only partially successful in treating hypertension. Locally applied ablation methods i.e. with radiofrequency catheters require an external expensive generator, and training in using electrophysiology equipment, which can restrict the use of those methods.

Hence there is a need for an improved device, system and/or method for treating hypertension.

### SUMMARY OF THE INVENTION

Accordingly, the present invention seeks to mitigate, alleviate or eliminate one or more of the above-identified deficiencies and to provide a new device, and kit of devices, suitable for a method for treatment of disorders to the blood pressure regulation system of the kinds referred to, according to the appended independent claims.

For this purpose a tissue cutting device according to claim 1 is provided, wherein the device is of an at least partly cylindrical shape and structured and arranged to be inserted in a temporary delivery shape through the vascular system into a renal artery and to be subsequently subjected to a change of shape, from said temporary delivery shape via an expanded delivered shape to a further expanded shape, extending at least beyond an inner surface of said tissue, in order to create cutting action configured for cutting said tissue and/or said body vessel.

In the following methods and devices are presented which act by cutting the nerves slowly through. The minimally invasive implantable devices can be delivered through state of the art delivery catheters as known from stenting, and do not require external generators, thereby being easily to introduce in normal equipped radiology catheter labs.

Advantageous features of the invention are defined in the dependent claims.

### BRIEF DESCRIPTION OF THE DRAWINGS

The invention will now be described in further detail by way of example under reference to the accompanying drawings, in which:

FIG. 1 shows an Aorta 10, a renal artery 101, and kidney 102;

FIG. 2 shows a perpendicular cut through a renal artery 101, showing the renal nerves 202 running in primarily in the adventitia of the vessel wall 201;

FIG. 3A shows a cylindrical tissue cutting device 300 seen from the end according to an embodiment of the invention;

FIG. 3B shows the same tissue cutting device seen from the side;

FIG. 3C shows the tissue cutting device seen from the side;

FIG. 3D shows a tissue cutting device with closed cells;

FIG. 4A shows the tissue cutting device in a first, temporary shape;

FIG. 4B shows the device in a second temporary shape;

FIG. 4C shows the tissue cutting device in a third, permanent shape;

FIG. 5A shows the device in the second shape just after release of the device into the vessel;

FIG. 5B shows the device in its permanent shape after cutting the nerves through;

FIG. 6A shows a cutting device where sections are connected by one or more long bridges, the device being in its second shape just after release into the vessel;

FIG. 6B shows a cutting device where sections are connected by one or more long bridges, the device being in a third permanent shape after cutting through the nerves;

FIG. 7 shows a cutting device where the device sections are designed to obtain different final shapes; and

FIG. 8 shows a self-expandable cutting device where the cutting section is restricted/held back by bioresorbable suture winded around the said section.

### DETAILED DESCRIPTION

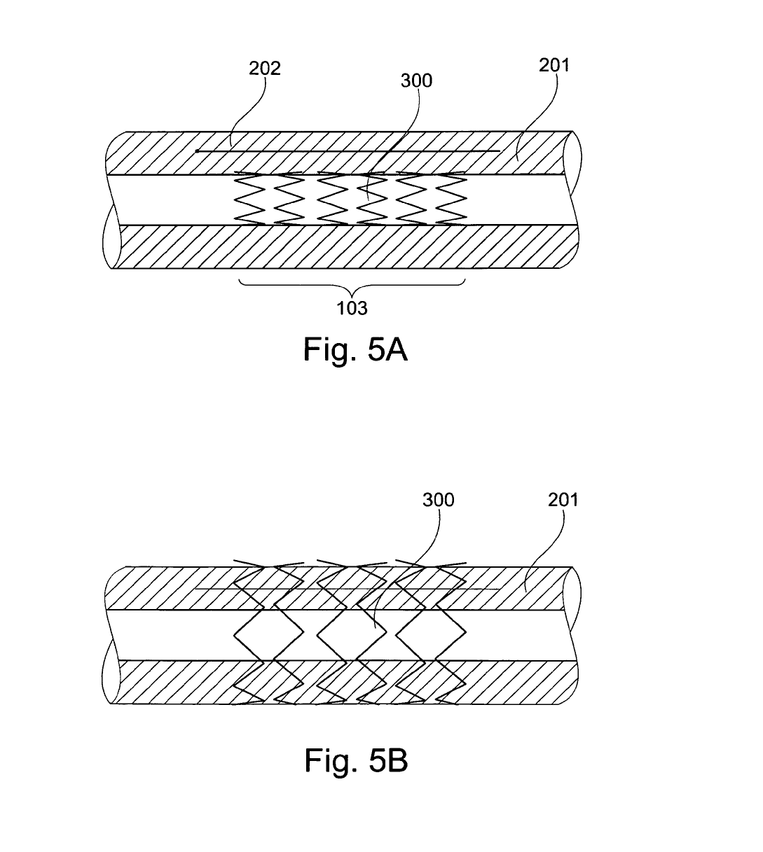

The cutting device 300 is inserted in its temporary shape in a desired position within the blood vessel. As a response to a stimulus, e.g. the body temperature, or by the elasticity of the device the cutting device will then strive towards changing its shape and obtaining the permanent shape. The memorized, permanent shape of the cutting device will not fit into the blood vessel. Thanks to this oversizing of the device, the cutting device will slowly force itself through surrounding tissue for obtaining the permanent shape, as shown in FIG. 5B. In FIG. 5b, the bridges 302 have for simplicity not been shown.

In this way, the cutting device will first penetrate the vessel wall from the inside of the vessel and thereafter tissue surrounding the blood vessel. Tissue cells that are penetrated will be killed, which will start a healing reaction in the body. Where the cutting device is placed in a desired position to change shape through tissue containing nerves, the nerve cells that are able to transmit electrical signals may thus be killed. The healing process will not restore the ability to transmit electrical signals and, therefore, the cutting device will reduce the ability of transmitting electrical signals through cut nerve tissue, for instance the sympathetic nerves.

In this manner, a target treatment of nerves adjacent to renal arteries is provided.

An example of a shape memory material is Nitinol, which is an alloy composed of nickel (54-60%) and titanium. Small traces of chrome, cobalt, magnesium and iron may also be present. This alloy uses a martensitic phase transition for recovering the permanent shape.

Shape memory materials may also be formed of shape memory polymers, wherein the shape-memory effect is based on a glass transition or a melting point. Such shape memory polymers may be produced by forming polymers of materials or combinations of materials having suitable properties.

For example, a shape memory polymer may be created of oligo(e-caprolactone) dimethacrylate combined with n-butyl acrylate. Also, biodegradable or bioresorbable materials may be used for forming these shape memory polymers. In this way, the cutting device may be designed such that it will be degraded or absorbed by the body after it has performed its change of shape. For example, a polylactic acid polymer and/or a polyglycolic acid polymer, poly (e-caprolactone) or polydioxanone may be used for forming a shape memory polymer that is biodegradable. A special feature of the resorbable shape memory polymers is that these will disappear from the tissue after having had its function, limiting potential negative effects of otherwise remaining polymer or Nitinol materials, such as perforations and damage to other adjacent tissues.

The cutting device may alternatively be formed to exhibit an elasticity such that it strives towards its permanent shape. The device may have a helical, spiral shape. The device may be made of a resilient material, shape or arrangement, e.g. a stainless steel or a magnesium alloy which in addition is biodegradable.

Optionally a cutting device may be designed to be expanded to its final permanent shape at delivery (e.g. by a balloon). The cutting forces thereby exerted by the expanded vessel wall striving to recover its original diameter.

Alternatively, or in addition, in some embodiments, a cutting balloon may be used to penetrate the vessel from the inside and cut through the wall tissue and surrounding tissue. In this manner adjacent nerve tissue may be cut through. The cutting balloon may have arranged thereon sharp blades. In this manner, the balloon does not have to penetrate into the vessel wall. Sharp projections from the balloon provide for the cutting action. The balloon may have a central lumen itself to allow for continued blood flow through the vessel during the cutting process. The projections may be releasable from the balloon. In this manner, the balloon may be retracted before the cutting action is finalized or before necrosis has occurred. The projections may be provided in form of hooks or barbs. The projections may have releasable connections to the balloon that break when the balloon is deflated, e.g. against the counter force of anchor units entered into the vessel wall tissue.

In some embodiments, the cutting balloon may instead of having sharp blades, have small spikes or spike-like protrusions that are pressed into the renal tissue. These spikes may be made of a swelling polymer. Thus, the spikes may swell and thereby induce signal blocking. The renal nerves may remain blocked for a few minutes. The spikes or spike-like protrusions are in some embodiments affixed to or forms parts of a balloon or balloon-like device.

In one embodiment, the spikes or spike-like protrusions are detachable from the balloon or balloon-like device. Therefore, after the spikes have been delivered at the target site, the spikes may be detached from the balloon or balloon-like device. Thereafter, the balloon or balloon-like device may be deflated, retracted and removed. Thus, stenosis caused by a permanent, short-term permanent or stationary balloon or balloon-like device can be avoided.

In some embodiments, the tissue cutting device is a device that creates scar tissue by transmural migration. Such a device may comprise a combination of either a biodegradable stent or stent-like device and a swelling polymer. The self-expanding stent or stent-like device may be made of e.g. magnesium or a magnesium alloy. The stent or stent-like device may either be balloon-expandable or self-expanding and placed in e.g. the renal artery or the pulmunary vein. If a balloon is used for expanding the stent or stent-like device, it may have a central lumen itself to allow for continued blood flow through the vessel during the process. The stent or stent-like device is in these embodiments covered at least partly externally with an expandable, and/or swelling polymer, which is adapted to migrate through the tissue over time. Once the polymer has migrated through the tissue, the stent or stent-like device will be exposed and will create scar tissue at the adventitia of the vessel wall 201 of e.g. a renal artery 101 and may thus e.g. block renal nerves. The renal nerves may remain blocked for a few minutes. Thus, e.g. renal denervation may be achieved.

In some embodiments, small spikes or spike-like protrusions are pressed into the renal tissue. These spikes may be made of a swelling polymer. Thus, the spikes may swell and thereby induce signal blocking. The spikes or spike-like protrusions are in some embodiments affixed to or forms parts of a stent or stent-like device. In one embodiment, the stent or stent-like device may be of metal.

In another embodiment, the stent or stent-like device may be of a biodegradable material. If the stent or stent-like device is biodegradable, then after the device has been degraded, only the spikes will remain. Thus, stenosis caused by a stent or stent-like device can be avoided.

In yet another embodiment, the spikes or spike-like protrusions are detachable from the stent or stent-like device. Therefore, after the spikes have been delivered at the target site, the spikes may be detached from the stent or stent-like device. Thereafter, the stent or stent-like device may be removed. Thus, stenosis caused by a stent or stent-like device can be avoided.

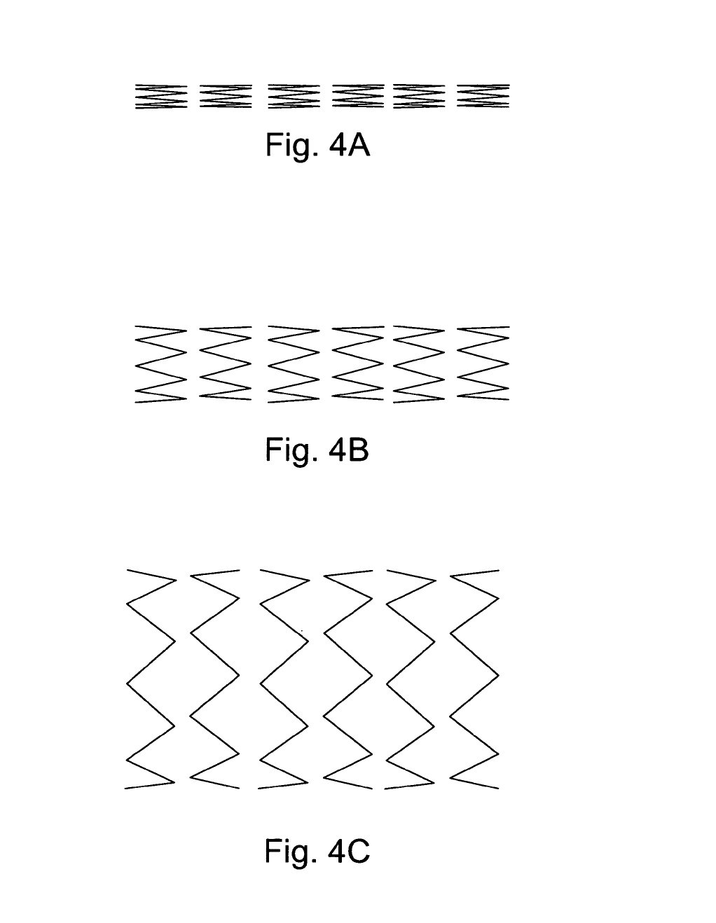

The cutting device may be tubular in both its temporary shape and its permanent shape, as shown in FIGS. 4-5. However, shape memory may be used for bringing the cutting device between any shapes. The shape of the cutting device in its first state is preferably compact as shown on drawing 4A to facilitate insertion of the cutting device through the vascular system. Thus, a tubular shape is suitable, but other shapes may be just as suitable. Further, the shape of the cutting device in its second state is designed such that the change of shape will provide penetration of specific tissue in order to block propagation of undesired nerve electrical signals. Also, the shape of the cutting device in its second state may be adjusted for fixing the cutting device to its desired position within the body.

The cutting device may be constructed of a net; i.e. its shape may comprise meshes or loops. This implies that a solid surface need not penetrate tissue, whereby the penetration through tissue and the forming of different shapes of the cutting device will be facilitated.

The edges of the cutting device facing the tissue to be penetrated may be made especially sharp to increase its effectiveness. Another feature is to cover the surface towards the tissue to be penetrated with drugs that increase the cutting effect or prohibit the thickening of the wall of the vessel in which the device is inserted.

In a polymeric resorbable cutting device the drug(s) can alternatively be fully or partly embedded in the material of the device. Examples of suited drugs are ciclosporin, taxiferol, rapamycin, tacrolimus, alcohol, glutaraldehyde, formaldehyde, and proteolytic enzymes like collagenase. Collagenase is effective in breaking down tissue and especially fibrin tissue, which is otherwise difficult to penetrate. Therefore, covering the surface of the cutting device with collagenase would particularly speed up the process of penetrating tissue. The drugs are attached to the surface of the cutting device according to well-known methods of attaching drugs to medical devices. One such method is embedding drugs into or under layers of polymers, which cover the surface. Of course, other methods may be used. Similarly, drugs preventing thrombosis and increasing in-growth of endothelium on the endothelial surface after penetration of the cutting device may be attached to the cutting device. Such drugs would be e.g. Endothelium Growth Factor, and Heparin.

To avoid that the vessel diameter expands during the shape change of the cutting device, thereby lowering the cutting action, the drug may alternatively or in addition be embedded or provided on the surface of the device and can also be vasoconstrictors as mentioned in the claims.

Preferably, the inside of the cutting device inserted into a blood vessel will be in contact with the blood stream inside the blood vessel. Such inside surface of the cutting device may as well be covered with antithrombotic drugs. Such drugs would be e.g. Heparin, Klopidogrel, Enoxaparin, Ticlopidin, Abciximab, and Tirofiban.

Cutting devices that are specifically suited for insertion into specific blood vessels will be described. All or some of these cutting devices may be delivered in a kit to be used for treatment of a blood pressure disorder . Alternatively, the cutting devices may be delivered separately.

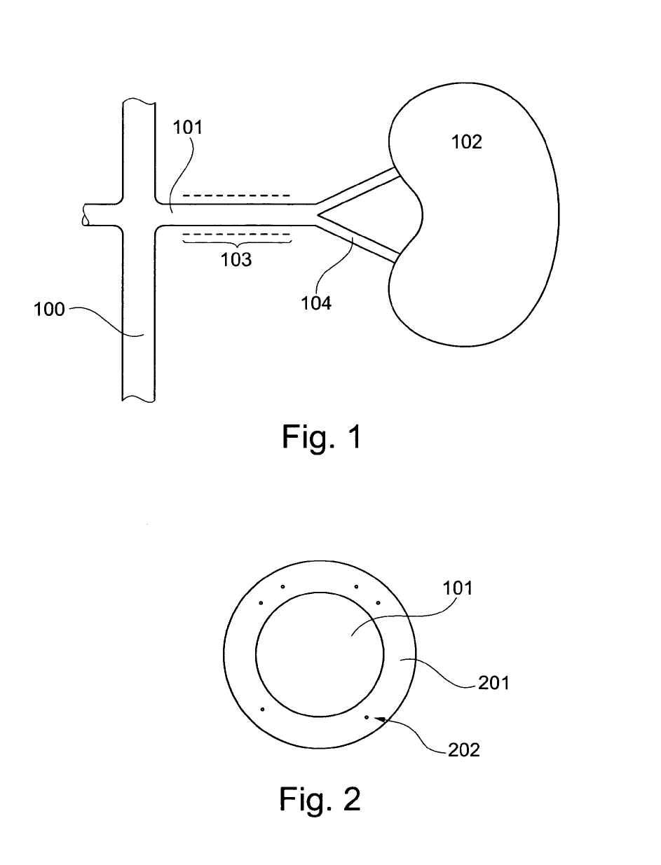

The devices described below are preferably intended to treat nerves 202 surrounding the renal arterias, mainly in the adventitia, 101 shown in FIG. 1. In FIG. 1, the typical cylindrical area to treat 103 is shown with dotted lines. Cutting devices can however be used wherever nerves are near a blood-path, accessible with catheters. The device is preferably inserted in the main part of the renal artery, but can optionally be used in the bifurcated part of renal arteries 104. The shape of the device hence may be tubular shape of a single cylindrical tubular unit. In addition, or in alternative embodiments, the tubular unit may have a bifurcated shape with one or several legs.

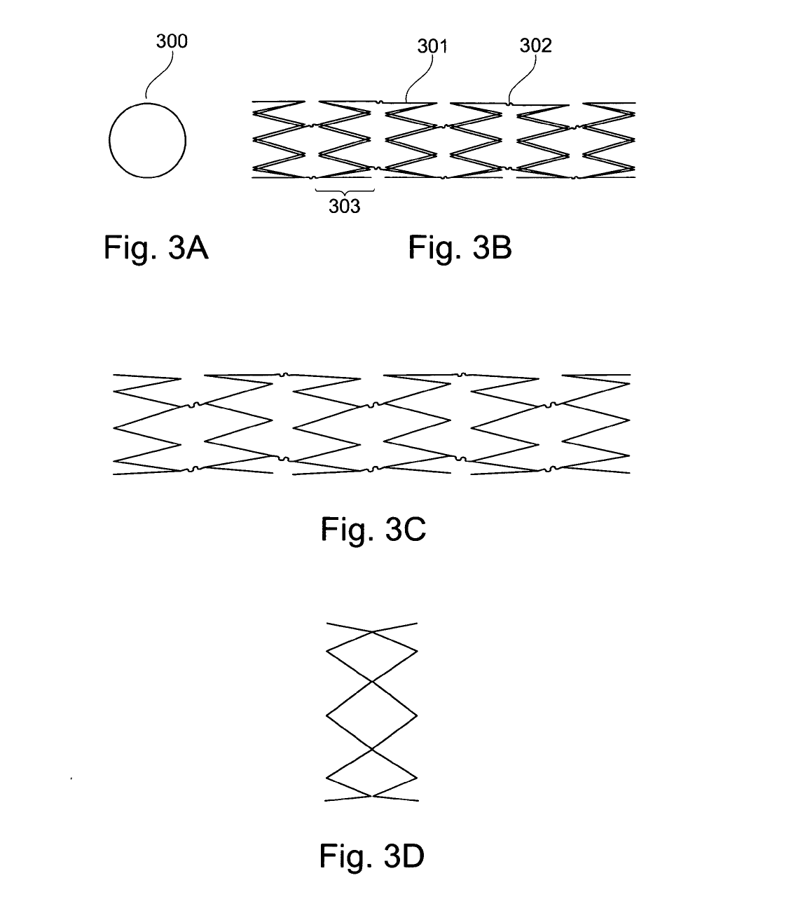

FIG. 3 shows a cylindrical cutting device. The device shown has a so-called open cell design, with one or more bridges (302) connecting the sections (303). The bridges shown are straight, but can alternative be curved or s-shaped to add flexibility of the bridge and device. FIG. 3A shows a cylindrical tissue cutting device 300 seen from the end according to an embodiment of the invention. FIG. 3B shows the same tissue cutting device seen from the side. By reference numeral 301 are referred as struts or wires, 302 as bridges, and 303 as sections. Both the wires in the front and in the back can be seen on the FIG. 3B. With the purpose of clarity, the wires in the back are not shown on FIG. 3C and in the following drawings.

Alternatively a closed or partly closed cell design could be used, where the sections are not parted. Closed cells are shown on FIG. 3D. With the purpose of clarity, the wires in the back are not shown on FIG. 3C and in the following drawings.

FIG. 4A shows the tissue cutting device in a first, temporary shape, typically when mounted in a delivery catheter. FIG. 4B shows the device in a second temporary shape just after releasing from the delivery catheter into the vessel, and FIG. 4C shows the tissue cutting device in a third, permanent shape, after the cutting action has taken place. (The bridges 302 have for simplicity not been shown).

FIG. 5A and B show a renal artery, with a sympathetic nerve 202, a vessel wall 201 and a cutting device 300. Only one nerve has been shown. In practice there may be more than one nerve. The vessel wall 201 however contains several nerves running along the vessel. A cylindrical part (preferably selected by the physician) in a suitable size of the vessel to be treated is indicated as 103. FIG. 5A shows the device in the second shape just after release of the device from the delivery catheter into the vessel. FIG. 5 B shows the device in i's permanent shape after cutting through the nerves. (The bridges 302 have for simplicity not been shown).

The wire/strut pattern of the device shown on the figures is an example. More complex patterns to improve cutting effect, or vessel conformability can be selected.

The figures shows a device made up of sections, but devices with a continuous pattern can be designed as well. The transitions from strut to strut can be rounded to improve material fatigue properties. The cutting device can alternatively be made as a braid.

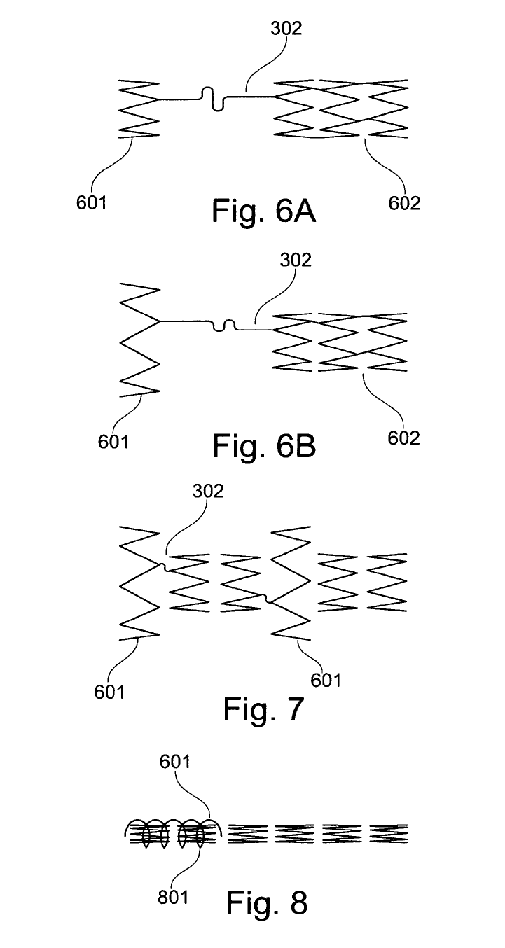

Optionally not all of the device length is intended for cutting. If the expansion area is long there will be danger of vessel rupture. FIG. 6 shows a cutting device where sections are connected by one or more long bridges. FIG. 6A shows the device in its second shape just after release into the vessel. FIG. 6B shows the device in a third permanent shape after cutting through the nerves. A section 602 act as stabilizer “anchor” and a larger section 601 acts as cutting section.

The anchor may in some embodiments be a stent. The stent does not grow into the vessel wall tissue.

The cutting section may in particular embodiments be an oversized stent specifically adapted to grow into the tissue. Care has to bee taken that a stent usually used to keep a vessel passage open and supporting a wall does not damage the vessel such that it ruptures. Another issue is that stents that are oversized tend to turn into the shape of an “8” inside the vessel and do not grow into the vessel wall, but restrict or even occlude flow in the vessel in an undesired way.

The device can have several cutting sections, and several stabilizing sections. FIG. 7 shows a device in its final expanded shape with two cutting sections. It can also be chosen to make the different sections of different materials, i.e. the cutting section of metal and the stabilizing (anchor) section of a bioresorbable polymer.

It can be of advantage that the device is securely embedded and fixed in the vessel endothelic layer before the cutting action starts. FIG. 8 shows a cutting device where the cutting section is hold back by bioresorbable suture winded around the said section. The bridges (302) have for simplicity not been shown. The device is implanted and after days or weeks when the remaining part of the device has attached to the vessel wall, the bioresorbable suture degrades and releases the cutting action. A similar effect can be obtained if the cutting section is temporarily fixed by bioresorbable adhesive or a bioresorbable tube surrounds the cutting section. If a stronger retaining force is needed a magnesium (resorbable) tube or wire can be used for this purpose.

Now, a system for delivery of a cutting device into a desired position in a blood vessel renal artery will be described. Each cutting device may be inserted into its desired position using such a delivery system. The delivery system allows a precise placement of each cutting device into the heart and the big vessels of the body. The delivery system has a restraining device, which keeps the cutting device in its temporary shape. This allows insertion into the blood vessel through catheters having a small bore, making minimal trauma to the patient. The restraining device may be a restraining tube, into which the cutting device is forced in its temporary shape. By cooling the cutting device, in case of a cutting device made of Nitinol, it may be easier to force the cutting device into the restraining tube. Once inserted into the desired position, the cutting device may be pushed out of the restraining tube by means of a piston or the cutting device may be released by retracting the restraining tube from its position over the cutting device. In case of a cutting device made of Nitinol, the cutting device may also be restrained by cooling to prevent it from obtaining a transition temperature trigging the change of shape. Thus, the cutting device may be restrained by cooling during insertion into the desired position and released by suspension of the cooling when inserted at the desired position. In WO 03/022179, such a delivery system is described in more detail. WO 03/022179 is incorporated herein by reference in its entirety for all purposes.

The delivery system used is preferably suited for an over the wire procedure, meaning suited for a guide wire.

Now, a method for treating a patient having a hypertension disorder will be described. The patient is prepared for operation and operation is performed in an environment allowing visualization of the renal artery and the attached big vessels using fluoroscopy and ultrasound according to conventional techniques.

The operation is started by making a puncture of an artery providing an access point to the vascular system of the patient according to conventional techniques. Usually, the femoral artery in the groin is used. However, other smaller arteries may be used instead. A delivery system is used for inserting the above described cutting device(s) into the renal artery.

The device delivery catheter may comprise an outer, restraining part, which covers the cutting device and keeps it in a contracted, temporary state. The restraining part may be axially displaceable in relation to the inner part. Thus, the restraining part may be retracted for releasing the cutting device.

The device delivery catheter can also incorporate a balloon to expand or partly expand the cutting device.

It should be emphasized that the preferred embodiments described herein is in no way limiting and that many alternative embodiments are possible within the scope of protection defined by the appended claims.

1. A tissue cutting device configured for reducing or removing undesired signal transmission in the human nerve system by cutting or migrating through the tissue incorporating the nerves at one or more positions,
wherein the device is structured and arranged to be inserted in a temporary delivery shape through the vascular system into a body vessel adjacent to the nerve or nerves and to be subsequently subjected to a change of shape via an expanded delivered shape to a further expanded shape, locally passing at least beyond the nerve(s) to be treated, in order to create cutting action configured for cutting said body vessel tissue.

2. The tissue cutting device as claimed in claim 1, wherein the device is structured and arranged to penetrate by said cutting action through a wall of said vessel, the wall containing the nerves to be treated.

3. The tissue cutting device as claimed in claim 1, wherein the device has an initial elongate shape and wherein the device is structured and arranged to change shape from the temporary delivery shape to expand its dimensions in a direction transversally to its elongate direction to the expanded delivered shape and further to the expanded shape.

4. (canceled)

5. The tissue cutting device as claimed in claim 1, wherein the device comprises a transversely expandable tubular part.

6. The tissue cutting device as claimed in claim 5, wherein said tubular part of the device is funnel-shaped.

7. The tissue cutting device as claimed in claim 5, wherein said tubular part comprises at least two axially separated tubular portions, which are interconnected by a connecting member.

8. The tissue cutting device as claimed in claim 7, wherein said tubular portions are transversely expandable to different degrees.

9. (canceled)

10. The tissue cutting device as claimed in claim 1, wherein an outside surface of the device struts or wires is provided with sharp edges along the struts or wires to improve the cutting effect.

11-17. (canceled)

18. The tissue cutting device as claimed in claim 1, wherein said device has a net-like shape formed of closed loops.

19. (canceled)

20. The tissue cutting device as claimed in claim 1, wherein the device is made of a shape memory polymer.

21. (canceled)

22. The tissue cutting device as claimed in claim 1, wherein the device is made of Nitinol.

23. The tissue cutting device as claimed in claim 1, wherein the device is made of stainless steel, a titanium alloy or a magnesium alloy.

24. (canceled)

25. The tissue device as claimed in claim 1 wherein sections or elements are adapted for cutting action.

26. The tissue device as claimed in claim 25 where the cutting sections or elements have a final larger cylindrical diameter than the remaining device, thereby exerting more cutting pressure than the remaining device.

27. The tissue device as claimed in claim 25 where the wires or struts in the cutting sections or elements are stiffer than in the remaining device, thereby exerting more cutting pressure than the remaining device.

28. (canceled)

29. The tissue device as claimed in claim 27 where the wires or struts in the cutting sections or elements are thinner or less wide than the remaining device, thereby exerting more cutting pressure than the remaining device.

30. The tissue cutting device as claimed in claim 1, wherein sections or elements of the device are starting the expansion later than other parts of the device.

31. The tissue cutting device as claimed in claim 30 where the later expansion mentioned is obtained by keeping the said sections on elements temporarily fixed with releasable connection elements, such as bioresorbable glue or bioresorbable suture which become resorbed upon implantation of the device.

32. The tissue cutting device as claimed in claim 1, comprising:
a biodegradable stent or stent-like device and
a swelling and/or expandable polymer adapted to gradually migrate through the tissue.

33-38. (canceled)

39. A method for treatment of disorders in the blood pressure regulation system, said method comprising: inserting a tissue cutting device through the vascular system to a desired position in a body vessel, and providing a change of shape of the tissue cutting device at said desired position to tissue in or adjacent said body vessel.

40. The method according to claim 39, wherein said tissue cutting device is inserted into a desired position in a renal artery.

41. The method according to claim 40, further comprising inserting another tissue cutting device to another of the desired positions.

42. (canceled)

43. The method according to claim 39, further comprising restraining the tissue cutting device in an insertion shape during the inserting of the tissue cutting device.

44-45. (canceled)

46. The method according to claim 43, further comprising releasing a restrain on the tissue cutting device when it has been inserted into the desired position for allowing said change of the shape of the tissue cutting device.

47. The method according to claim 39, wherein said tissue cutting device comprises a stent or stent-like device having spikes or spike-like protrusions and said method further comprising
providing said change of shape of said tissue cutting device by expanding said stent or stent-like device to an expanded delivered shape and bringing said cutting device to a further expanded shape by expanding said spikes or spike-like protrusions.

48. (canceled)

49. The method according to claim 47, further comprising:
detaching said spikes or spike-like protrusions from said stent or stent-like device;
bringing said stent or stent-like device into a temporary delivery shape and
retracting and/or removing said stent or stent-like device.

50. The method according to claim 39, wherein said tissue cutting device comprises a balloon or balloon-like device having spikes or spike-like protrusions and said method further comprising providing said change of shape of said tissue cutting device by expanding said balloon or balloon-like device to an expanded delivered shape and bringing said cutting device to a further expanded shape by expanding said spikes or spike-like protrusions.

51. The method according to claim 47, further comprising:
detaching said spikes or spike-like protrusions from said balloon or balloon-like device;
bringing said balloon or balloon-like device into a temporary delivery shape and
retracting and/or removing said balloon or balloon-like device.

52-58. (canceled)
